(19) United States
(12) Patent Application Publication
(10) Pub. No.: US 2011/0200558 A1
(43) Pub. Date: Aug. 18, 2011

(54) Treatment or Prevention of Hepatitis C with Immunomodulator Compounds

(75) Inventors: Israel Rios, Menlo Park, CA (US); Cynthia W. Tuthill, Menlo Park, CA (US)

(73) Assignee: SCICLONE PHARMACEUTICALS, INC., Foster City, CA (US)

(21) Appl. No.: 13/057,745

(22) PCT Filed: Aug. 4, 2009
(86) PCT No.: PCT/US09/52666

§ 371 Date: Apr. 15, 2011

Related U.S. Application Data
(60) Provisional application No. 61/086,654, filed on Aug. 6, 2008.
(60) Provisional application No. 61/167,363, filed on Apr. 7, 2009.

(51) Int. Cl. A61K 38/21; A61K 38/05; A61P 31/14

(52) U.S. Cl. 424/85.7; 424/85.4; 514/4.3

(57) ABSTRACT

A method of treatment for treating or preventing hepatitis C (HepC) in a target subject, including administering to the target subject an effective amount of an immunomodulator compound of Formula A

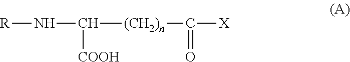

wherein, n is 1 or 2, R is hydrogen, acyl, alkyl or a peptide fragment, and X is an aromatic or heterocyclic amino acid or a derivative thereof, wherein;
(i) the immunomodulator compound is administered to the subject at a dosage of greater than 0.001 mg/kg;
(ii) the immunomodulator compound is administered in a combination treatment regimen further including administration to the subject of ribavirin, wherein the immunomodulator compound and the ribavirin are administered to the subject separately or together in the treatment regimen;
(iii) the immunomodulator compound is administered in a combination treatment regimen with a specifically targeted antiviral therapy for hepatitis C (STAT-C) agent, wherein the immunomodulator compound and the STAT-C agent are administered to the subject separately or together in the treatment regimen; or
(iv) a combination of at least two of (i), (ii), and (iii).

### BACKGROUND OF THE INVENTION

1. Field of the Invention

The present invention relates to the field of Hepatitis C treatment.

2. Description of the Background Art

Hepatitis C is a blood-borne, infectious, viral disease that is caused by the hepatotropic virus Hepatitis C virus (HCV). The infection can cause liver inflammation, and chronic hepatitis can result in cirrhosis (fibrotic scarring of the liver) and liver cancer.

The hepatitis C virus (HCV) is generally spread by blood-to-blood contact with an infected person's blood. An estimated 150-200 million people worldwide are infected with hepatitis C. Current treatment is a combination of pegylated interferon alpha and the antiviral drug ribavirin for a period of 24-48 weeks.

Three major classes of interferons have been described for humans, Type I, type II and type III, classified based on the type of receptor through which they signal. Human type I IFNs comprise a vast and growing group of IFN proteins, whereas types II and III presently are much smaller groups.

Interferons in general have several effects in common. They are antiviral and possess antioncogenic properties, macrophage and natural killer lymphocyte activation, and enhancement of major histocompatibility complex glycoprotein classes I and II, and thus presentation of foreign (microbial) peptides to T cells. In a majority of cases, the production of interferons is induced in response to microbes such as viruses and bacteria and their products (viral glycoproteins, viral RNA, bacterial endotoxin, bacterial flagella, CpG DNA), as well as mitogens and other cytokines, for example interleukin 1, interleukin 2, interleukin-12, tumor necrosis factor and colony-stimulating factor, that are synthesised in the response to the appearance of various antigens in the body. Their metabolism and excretion take place mainly in the liver and kidneys. They rarely pass the placenta and the blood-brain barrier.

Several different types of interferon are now approved for use in humans, and interferon therapy is used (in combination with chemotherapy and radiation) as a treatment for many cancers. When used in the systemic therapy, IFN-alpha and IFN-gamma are mostly administered by a subcutaneous injection. The injection of interferons in the muscle, in the vein, or under skin is generally well tolerated. The most frequent side-effects are flu-like symptoms: increased body temperature, feeling ill, fatigue, headache, muscle pain, convulsion, dizziness, hair thinning, and depression. Erythema, pain and hardness on the spot of injection are also frequently observed. All known effects are usually reversible and disappear a few days after the therapy has been finished. However, there are some serious side effects and the patient is advised to read the accompanying pamphlet.

More than half of hepatitis C patients treated with interferon respond with better blood tests and better liver biopsies. There is some evidence that giving interferon immediately following infection can prevent hepatitis C; however, people infected by hepatitis C often do not display symptoms of HCV until months or years later.

More recently, the FDA approved pegylated interferon-alpha, in which polyethylene glycol is added to make the interferon last longer in the body. (Pegylated interferon alpha-2b was approved in January 2001; pegylated interferon alpha-2a was approved in October 2002.) The pegylated form is injected once weekly, rather than three times per week for conventional interferon-alpha. Used in combination with the antiviral drug ribavirin, pegylated interferon produces sustained cure rates of 75% or better in people with genotype 2 or 3 hepatitis C (which is easier to treat) but still less than 50% in people with genotype 1 (which is most common in the U.S. and Western Europe).

There remains a need in the art for improvements in methods for treating or preventing hepatitis C (HepC).

### SUMMARY OF THE INVENTION

In accordance with the present invention, a method of treatment for treating or diminishing hepatitis C (HepC) infection in a target subject, comprising administering to said target subject an effective amount of an immunomodulator compound of Formula A

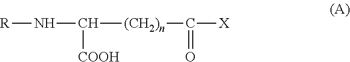

wherein, n is 1 or 2, R is hydrogen, acyl, alkyl or a peptide fragment, and X is an aromatic or heterocyclic amino acid or a derivative thereof, wherein;
(i) said immunomodulator compound is administered to said subject at a dosage of greater than 0.001 mg/kg;
(ii) said immunomodulator compound is administered in a combination treatment regimen further comprising administration to said subject of ribavirin, wherein said immunomodulator compound and said ribavirin are administered to the subject separately or together in the treatment regimen;
(iii) said immunomodulator compound is administered in a combination treatment regimen with a specifically targeted antiviral therapy for hepatitis C (STAT-C) agent, wherein the immunomodulator compound and the STAT-C agent are administered to the subject separately or together in the treatment regimen; or
(iv) a combination of at least two of (i), (ii), and (iii).

### DESCRIPTION OF THE PREFERRED EMBODIMENTS

Immunomodulator compounds for use in accordance with the present invention, comprise immunomodulators of Formula A:

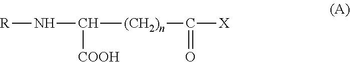

In Formula A, n is 1 or 2, R is hydrogen, acyl, alkyl or a peptide fragment, and X is an aromatic or heterocyclic amino acid or a derivative thereof. In certain embodiments, X is L-tryptophan or D-tryptophan. Appropriate derivatives of the aromatic or heterocyclic amino acids for “X” are: amides, mono- or di-(C₁-C₆) alklyl substituted amides, arylamides, and (C₁-C₆) alkyl or aryl esters. Appropriate acyl or alkyl moieties for “R” are: branched or unbranched alkyl groups of 1 to about 6 carbons, acyl groups from 2 to about 10 carbon atoms, and blocking groups such as carbobenzyloxy and t-butyloxycarbonyl. In certain embodiments, the carbon of the CH group shown in Formula A has a stereoconfiguration, when n is 2, that is different from the stereoconfiguration of X.

Certain embodiments utilize compounds such as γ-D-glutamyl-L-tryptophan, γ-L-glutamyl-L-tryptophan, γ-L-glutamyl-Nᵢₙ-formyl-L-tryptophan, N-methyl-γ-L-glutamyl-L-tryptophan, N-acetyl-γ-L-glutamyl-L-tryptophan, γ-L-glutamyl-D-tryptophan, β-L-aspartyl-L-tryptophan, and β-D-aspartyl-L-tryptophan. Particularly preferred embodiments utilize γ-D-glutamyl-L-tryptophan, sometimes referred to as SCV-07. These compounds, methods for preparing these compounds, pharmaceutically acceptable salts of these compounds and pharmaceutical formulations thereof are disclosed in U.S. Pat. No. 5,916,878, incorporated herein by reference.

Included are biologically active analogs having substituted, deleted, elongated, replaced, or otherwise modified portions which possess bioactivity substantially similar to that of SCV-07, e.g., an SCV-07 derived peptide having sufficient homology with SVC-07 such that it functions in substantially the same way with substantially the same activity as SCV-07.

SCV-07, γ-D-glutamyl-L-tryptophan, is a member of a class of immunomodulatory drugs that possess γ-glutamyl or β-aspartyl moieties, which was discovered by Russian scientists and is being examined for efficacy in several indications in the U.S. by SciClone Pharmaceuticals, Inc.

SCV-07 possesses a number of immunomodulatory activities in vivo and in vitro. SCV-07 increases Con-A-induced thymocyte and lymphocyte proliferation, increases Con-A-induced interleukin-2 (IL-2) production and IL-2 receptor expression by spleen lymphocytes, and stimulates expression of Thy-1.2 on bone marrow cells. In vivo, SCV-07 has a strong immunostimulatory effect on 5-FU-immune-suppressed animals and in a model of immunization with sheep red blood cells. Recent information on the subcellular mechanism of action, namely an inhibition of STAT-3 dependent signaling and the consequent effects of decreasing IL-10 and T regulatory cells, also support that SCV-07 would be useful for treatment of hepatitis C virus infection.

Due to the nature of the hepatitis C virus, it appears that different, higher doses of SCV-07 may be required for efficacy, as compared to treatment of other viral infections. Other viral infections results in small, localized, or “contained” infections, whereas infection with the hepatitis C virus results in high quantities of viral particles in the circulation, and therefore higher concentrations of SCV-07 appear to be required to fight this more generalized infection. Using higher doses of SCV-07 to fight hepatitis C virus can be shown over a short duration, where a treatment with one to two weeks of SCV-07 alone may result in a decrease in the circulating viral concentrations

In certain embodiments, the Formula A compounds may be administered as dosages in the range of from greater than 0.07 mg, up to about 700 mg. In certain embodiments, the compounds of Formula A are administered at a dosage within a range of about 0.07-700 mg, at a dosage of about 0.7-70 mg, or at a dosage of about 7-70 mg.

Dosages may be administered one or more times per week, e.g., on a daily basis, with dosages administered one or more times per day. In certain embodiments, dosages are administered twice weekly. Administration can be by any suitable method, including orally, nasally, transdermally, sublingually, by injection, periodic infusion, continuous infusion, and the like. The dosages may be administered by subcutaneous injection and intramuscular injection, although other forms of injection and infusion may be utilized, and other forms of administration such as oral or nasal inhalation or oral ingestion may be employed.

Dosages may also be measured on a milligrams per kilogram basis, with dosages in the range of from greater than 0.001 mg/kg up to about 10 mg/kg, dosages within the range of from greater than about 0.01 mg/kg up to about 10 mg/kg, or dosages of about 0.1-2 mg/kg.

In certain embodiments, the addition of ribavirin to treatment with SCV-07, or other Formula A compounds, are included. Accordingly, in certain embodiments, the Formula A compound is administered in a combination treatment regimen further comprising administration to the subject of another agent which is active against HCV, e.g., ribavirin. Even low doses of the Formula A compounds (i.e., 0.001 mg/kg or 0.07 mg or lower) may work in combination with ribavirin (or any other agent in a combination regimen). Ribavirin dosages may be within a range of about 100-2000 mg/day, about 800-1200 mg/day, or about 1000-1200 mg/day. Ribavirin dosages may be administered one or more times per week, e.g., on a daily basis, with dosages administered one or more times per day.

In certain embodiments e.g., in embodiments in which the Formula A compound is utilized as a sole therapy for treatment or prevention of HCV infection, a higher dosage of greater than 0.001 mg/kg of the Formula A compound is utilized. For sole therapy treatment, dosages within a range of 0.002-1 mg/kg may be utilized, e.g., within a range of 0.002-0.1 mg/kg, or 0.002-0.01 mg/kg. According to one embodiment, a dosage within a range of from greater than about 0.01 mg/kg up to about 10 mg/kg is utilized in a sole therapy, e.g., about 0.1-2 mg/kg.

In certain embodiments, an SCV-07 dose of 0.1-2.0 mg/kg daily for up to 48 weeks is utilized.

Actual dosages per day of administration of a Formula A compound in a sole therapy may be within a range of about 0.2-200 mg, 0.2-20 mg, or 0.2-2 mg.

In accordance with one embodiment, the present invention relates to treatment of hepatitis C (HCV) infection by administering an immunomodulator compound to a mammalian subject, preferably a human patient, wherein said immunomodulator compound is administered to said subject in a treatment regimen without administering an interferon (IFN) to said subject.

One embodiment of the present invention is treatment of HCV infection in a treatment regimen consisting essentially of administering the immunomodulator compound to the subject.

Type 1 Interferon alpha (IFN-a) is thought to clear hepatitis C infection through both antiviral and immune modulating mechanisms. IFN-a has potent antiviral activity, but does not act directly on the virus or replication complex; rather it acts by inducing IFN-stimulated genes which establish an intracellular state which decreases the replication of the virus. Hundreds of genes are stimulated by IFN-a, many related to this anti-viral activity such as 2′,5′-oligoadenylate synthetase (OAS), which triggers ribonuclease-L activation and subsequent degradation of viral RNA. Other genes that are activated are intimately involved in the immune response, especially those promoting a Th1 phenotype which is associated with greater clearance of hepatitis C.

It is thought that exogenously added IFN-a acts in the same way as endogenous IFN-a, binding to IFN-a receptors and causing second messengers to translocate to the nucleus to stimulate the interferon-sensitive genes and thereby activating both anti-viral and immune mechanisms. However, direct administration of IFN-a results in multiple side effects that can limit treatment, and that are possibly related to the use of a single cytokine in high concentrations. A broader stimulation of immunity may avoid this excessive exposure to any single cyokine and thereby achieve the benefit of a stimulated immune response and indirect antiviral activity with fewer and more tolerable side effects.

The dipeptide γ-Glu-Trp, SCV-07, has demonstrated a broad spectrum of immune stimulation, enhancing the Th1 subset of T cells and increasing antigen-specific T cell responses. Treatment with SCV-07 increases production of the Th1 cytokines IFN-g and IL-2, while decreasing the Th2 cytokines IL-4 and IL-10. SCV-07 treatment has led to increased survival in several preclinical models of viral infection, including Pichinde virus in guinea pigs and Cottontail papilloma virus in rabbits, and to decreased recurrence in guinea pigs infected with herpes virus HSV-2. By virtue of these immune-stimulating and anti-viral activities, in certain embodiments SCV-07 can replace IFN-a in the treatment of hepatitis C.

A short treatment with SCV-07 may lead to decreased viral replication and increased markers of Th1 immunity. Due to stimulation of the Th1 pathway, endogenous IFN-a may increase, and markers of IFN-a action such as OAS may also be measurably increased.

A treatment regimen in accordance with one embodiment of the invention includes administration to the subject of a specifically targeted antiviral therapy for hepatitis C (STAT-C) agent, wherein the Formula A compound and the STAT-C agent are administered to the subject separately or together in the treatment regimen.

Included in the invention are STAT-Cs that target two enzymes required for hepatitis C reproduction: serine protease and polymerase, STAT-Cs thus encompass hepatitis C protease and polymerase inhibitors.

Examples of protease inhibitors include SCH503034, VX-950 (Telaprevir), VX500, R7227, ITMN-191, ACH-1095 and TMC435350.

Examples of polymerase inhibitors include GS9190, GSK625433, R7128, R1626, VCH-759, MK-0608, IDX-184, A-837093, and AG-021541.

Certain protease inhibitors which may be used in the invention are further discussed below.

BILN-2061. The serine protease HCV NS3 mediates polyprotein processing and that has a shallow hydrophobic binding region. BILN-2061 (Boehringer Ingelheim, Ingelheim, Germany), a potent inhibitor of NS3, was one of the first HCV protease inhibitors tested. A phase 1 clinical trial assessing the antiviral efficacy of BILN-2061 showed that this agent rapidly reduced viral load within the first 48 hours. Although BILN-2061 demonstrated potent antiviral activity against HCV genotype 1, the virologic response was less pronounced and more variable in HCV genotype 2 and 3 patients.

VX-950 (Telaprevir, Vertex Pharmaceuticals, Cambridge, Mass.) is a selective, specific, and potent peptidomimetric inhibitor of the HCV NS3-4A serine protease. In a phase 1 clinical trial, patients with HCV genotype 1 were randomized to placebo or to VX-950 monotherapy administered at does of 450 mg or 750 mg every 8 hours or 1250 mg every 12 hours for 14 days. Results of this preliminary study showed that VX-950 administered at a dose of 750 mg every 8 hours resulted in a 4.4-log drop in HCV RNA from baseline. In fact, this reduction in viral load occurred within the first 4 days of treatment. A subsequent study examined efficacy during dosing with VX-950 in combination with pegylated interferon alfa-2a. In this study, one cohort of subjects received VX-950 in combination with pegylated interferon alfa-2a for 14 days. At the end of the 14 days of the study period, a 5.5-log decline in HCV RNA was noted. In fact, 6 of 8 patients had undetectable HCV RNA (<30 IU/mL) by day 14. After the completion of 14 days of the study period, in an off-study follow-on treatment, all patients were given a combination of pegylated inferferon alfa-2a plus ribavirin for another 24 weeks.

Of the 6 patients who completed 24 weeks of combination therapy with pegylated interferon alfa-2a and Ribavirin, 5 remained HCV RNA undetectable after 12 weeks of follow-up.

In another larger study with longer duration, in a double-blind, placebo-controlled fashion (phase 2b PROVE1 clinical trial), the safety and efficacy of VX-950 in combination with pegylated interferon alfa-2a and Ribavirin in treatment-naïve HCV genotype 1 patients was assessed. In this study, patients received at least 1 dose of VX-950 or placebo in addition to pegylated interferon alfa-2a ribavirin. An interim analysis presented during the 42^{nd} Annual Meeting of the European Association for the Study of the Liver (EASL) showed that 88% of patients receiving the triple combination regimen achieved a rapid viral response (HCV RNA <30 IU/mL0; 79% achieved a rapid viral response as measured by HCV RNA levels <10 IU/mL at week 4 of treatment. In comparison, 16% of patients who received the pegylated interferon alfa-2a, Ribavirin, and placebo combination achieved a rapid viral response (HCV RNA <30 IU/mL) and 11% achieved a rapid viral response as measured by HCV RNA <10 IU/mL. The virus remained undetectable after 20 weeks of follow-up (sustained virologic response “SVR 20”) in 6 of 9 patients who completed their treatment.

SCH503034 (Shering-Plough, Kenilworth, N.J.) is another NS3 serum protease inhibitor. In phase 1 clinical trial, 61 patients infected with HCV genotype 1 who were nonresponders to previous treatment with pegylated interferon alfa were randomized to receive SCH503034 (100 mg twice daily, 200 mg twice daily, 400 mg twice daily, 400 thrice daily) or placebo for 14 days. Preliminary results showed that after 14 days of monotherapy, SCH503034 given at a dose of 400 mg thrice daily was associated with an approximately 2-log drop in HCV RNA from the baseline value. A follow-up study assessed the virologic response of combination SCH503034 plus pegylated interferon alfa-2b in patients infected with HCV genotype 1 who were previous nonresponders to pegylated interferon alfa-2b-based therapy. Preliminary results suggested that the best viral response occurred when pegylated interferon alfa-2b was given in combination with SCH503034 400 mg thrice daily for 14 days; a 2.88-log reduction in HCV RNA occurred among patients in this treatment group.

Other Protease Inhibitors. The antiviral activity of ACH-806 (also known as GS-9132; Achillion Pharmaceuticals/Gilead Sciences), another HCV protease inhibitor, has been tested in phase 1 clinical trial. In data presented at an EASL meeting in Barcelona, Spain, treatment with ACH-806 was associated with a 2.38-log drop in HCV RNA within 5 days of therapy. Also, ITMN B (Intermune, Brisbane, Calif.) is another HCV protease inhibitor that has been tested in replication model(s).

Certain HCV RNA Polymerase Inhibitors which may be used in the invention are further discussed below.

NM283 (Valopicitabine, Idenix Pharmaceuticals, Cambridge, Mass.), is a ribonucleoside analog that targets the viral RNA polymerase and is a viral RNA chain terminator. A phase ½ dose-escalation trial of NM283 in the range of 50 mg per day to 800 mg per day has been conducted. Results showed that the best response to NM283 occurred at a dose titrated between 400 mg and 800 mg/day.

R1626 (Roche Pharmaceuticals, Basel, Switzerland), another nucleoside analog oral polymerase inhibitor, has been administered at doses ranging from 500 mg to 1500 mg twice daily for 14 days in treatment-naive HCV genotype 1 patients. The initial clinical trial involving this agent demonstrated a clinically significant approximately 1.2-log reduction in HCV RNA associated with the 1500-mg twice-daily dosing regimen. A subsequent multiple ascending dose study of R1626 (500 mg, 1500 mg, 3000 mg, and 4500 mg twice daily for 14 days) was conducted in previously untreated patients with HCV genotype 1 infection. Mean (median) HCV viral reductions of 0.3 (0.2), 1.2 (0.8), 2.6 (2.7), and 3.7 (4.1) log 10 were observed for doses of 500 mg, 1500 mg, 3000 mg, and 4500 mg, twice daily, respectively.

The nonnucleoside polymerase inhibitor HCV-796 (ViroPharma, Exton, Pa. and Wyeth Research, Philadelphia, Pa.) has been studied in a phase 1 clinical trial at doses ranging from 50 mg per day to 1500 mg per day. Approximately a 1.2-log drop in the HCV RNA viral load was observed among patients receiving the higher doses (500-1500 mg/day). In addition to these agents, several other polymerase inhibitors, including MK-0608, A-837093, and AG-021541, are also under development.

In certain embodiments, an STAT-C dose of 1000-3000 mg/day for up to 48 weeks is utilized.

When ribavirin is added to treatment with SCV-07, or other Formula A compounds, a STAT-C, may be included. Accordingly, in certain embodiments, the Formula A compound and STAT-C agent are administered in a combination treatment regimen further comprising administration to the subject of another agent which is active against HCV, such as ribavirin. Ribavirin dosages may be within a range of about 100-3000 mg/day, or about 800-3000 mg/day, or about 1000-3000 mg/day. Ribavirin dosages may be administered one or more times per week, e.g., on a daily basis, with dosages administered one or more times per day.

The dipeptide γ-Glu-Trp, SCV-07, has demonstrated a broad spectrum of immune stimulation, enhancing the Th1 subset of T cells and increasing antigen-specific T cell responses. Treatment with SCV-07 increases production of the Th1 cytokines IFN-g and IL-2, while decreasing the Th2 cytokines IL-4 and IL-10. SCV-07 treatment has led to increased survival in several preclinical models of viral infection, including Pichinde virus in guinea pigs and Cottontail papilloma virus in rabbits, and to decreased recurrence in guinea pigs infected with herpes virus HSV-2.

A short treatment with SCV-07 may lead to decreased viral replication and increased markers of Th1 immunity. Due to stimulation of the Th1 pathway, endogenous IFN-a may increase, and markers of IFN-a action such as OAS may also be measurably increased.

In accordance with one embodiment, the present invention relates to treatment of hepatitis C(HCV) infection by administering an immunomodulator compound to a mammalian subject, preferably a human patient, wherein said immunomodulator compound is administered to said subject in a combination regimen with at least one interferon (IFN). The Formula A compound also can be administered with other agents which are active against HCV.

The interferon may be selected from type I, II and/or III interferons. In certain embodiments, the interferon is an alpha interferon, a beta interferon, a gamma interferon or a combination thereof. In certain embodiments, the interferon is an alpha interferon.

In certain embodiments, the interferon is interferon alpha-2a, e.g., pegylated interferon alpha-2a, and/or interferon alpha-2b, e.g., pegylated interferon alpha-2b.

Interferon alpha-2a can be administered at dosages of from about 1-1000 ug, and can be administered daily, or one, two, three, four, five or six days per week, and can be administered multiple times per day. For example, 180 ug of pegylated interferon alpha-2a can be administered once weekly. Alternatively, pegylated interferon alpha-2a can be administered at lower dosages of 135 ug per week.

Suitable dosages of interferon alpha-2b are in a range of from about 0.5-10 million units (MU). For example, one dosage regimen of interferon alpha-2b is 3 MU pegylated interferon alpha-2b, three times weekly.

In some embodiments, the Formula A compound and/or STAT-C, and/or Ribavirin and/or interferon is/are present in a pharmaceutically acceptable liquid carrier, such as water for injection, saline in physiological concentrations, or similar, or in tablet form with suitable dry carrier(s) and excipient(s).

Effective amounts of Formula A compound, STAT-C, Ribavirin and/or interferon can be determined by routine dose-titration experiments.

### Example 1

### Treatment of Hepatitis C with the Immune-Stimulating Dipeptide SCV-07

SCV-07, g-D-Glu-Trp, is an immunomodulatory dipeptide which has been shown to increase T-cell differentiation and function and inhibit STAT3 activity, and is therefore hypothesized to be useful in treatment of infectious diseases associated with an inadequacy of Th1-mediated immunity such as hepatitis C (HCV).

Here we describe interim results from a phase 2a, proof of principle, multicenter, randomized, controlled, single-blind dose ranging study, which is evaluating the safety and antiviral effect of SCV-07 in non-cirrhotic subjects with genotype 1 chronic HCV infection. Subjects enrolled in the study must have relapsed to previous treatment with PEGylated interferon and ribavirin; therefore any change in HCV viral load of greater than 0.5 log could be considered a significant response.

Subjects are treated with 7 daily injections of SCV-07 (8 subjects per cohort) or saline (2 per cohort). Cohort 1 (0.01 mg/kg SCV-07) and Cohort 2 (0.10 mg/kg) have completed enrollment; no responses were seen in the lower dose but a decrease in viral load and increase in biomarker neopterin was seen in 2 subjects at the higher dose, on the day 14 follow up visit (see Table 1). Due to this apparent late response, a 30-day follow up visit was added for Cohort 3. Of the 4 subjects in this Cohort who have reached the 30-day visit, one has responded with a 1.2-log drop in HCV along with an increase in neopterin (see Table1). None of the subjects treated with saline had decreases in HCV of greater than 0.3 log, or increases of neopterin of greater than 3 nmol/L. No significant changes in ALT from baseline were seen in any of the subjects.

SCV-07 was well tolerated in all 3 dose groups. No SAEs were reported and no subject had to discontinue treatment due to AEs. The responses seen are encouraging, and the correlation of viral load changes with increases in neopterin suggest SCV-07 may be acting to prime the immune system to increase the possibility of development of a sustained viral response. Further clinical investigation into longer treatment duration or addition to other anti-viral agents appears warranted.

TABLE 1

| | | | | | HCV viral load | | Neopterin | |
|---|---|---|---|---|---|---|---|---|
| Cohort | Dose | Race | Gender | Age | (log10 decrease) | | (nmol/L) | |
| 1 | 2 saline | 10 white/ | 7 M, 3 F | 39-69 | Day 14 | Day 30 | Day 14 | Day 30 |
| | 8 SCV-07 | Caucasian | | | | | | |
| | 0.01 mg/kg | | | | | | | |
| 2 | 2 saline | 7 white/ | 6 M, 5 F | 48-59 | 0.9 | | 32 | |
| | 9 SCV-07 | Caucasian | | | 0.6 | | 12 | |
| | 0.10 mg/kg | 2 Latino/ | | | | | | |
| | | Hispanic | | | | | | |
| | | 2 Black/ | | | | | | |
| | | Africa | | | | | | |
| | | American | | | | | | |
| 3 | 4 SCV-07 | | 3 M, 1 F | | | 1.2 | | 33 |
| | 1.0 mg/kg | | | | | | | |
| | (interim) | | | | | | | |

An unexpected and surprising finding is the fact that the plasma concentration of neopterin increases in each patient that showed a viral response. A plasma concentration of neopterin above 10 nmol/L represents a “stimulated” immune system. This shows that SCV-07 is definitely stimulating the immune system. Increased levels of neopterin also are correlated with increased sustained viral responses against HCV, and so is very strong evidence for the use of SCV-07 in this disease. The timing of the increase is also of interest. We did not see any effects of SCV-07 until “late” (i.e. the dosing was on Days 1-7, but the decreases in HCV and increases in neopterin were not seen until Days 14 or 30). This suggests the immune system may be “primed” by treatment with SCV-07.

### Example 2

Hepatitis C patients treated as in Example 1 above also are administered a STAT-C agent at a dose of 1000-3000 mg/day for up to 48 weeks, and show an improved response against HCV.

### Example 3

Hepatitis C patients treated as in Examples 1 and 2 above also are administered ribavirin at a dose of 1000-2000 mg/day for up to 48 weeks, and show an improved response against HCV.

### Example 4

Hepatitis C patients are treated with SCV-07 at a dose of 0.1-2.0 mg/kg/day, and also are treated with STAT-C at a dose of 1000-3000 mg/day, and/or ribavirin at a dose of 1000-1200 mg/day, and/or 3MU pegylated interferon alpha-2b three times per week, and/or 180 ug pegylated interferon alpha-2a once per week, for up to 48 weeks, and show an improved response against HCV.

Treatment, as in the examples above, results in achieving a sustained virological response (SVR) (which is accepted as cure and used as the efficacy endpoint for FDA approval), of 65-75%. This compares favorably with the current SOC SVR of 45-50%. This improved SVR is achieved in a regimen that has much less toxicity when compared to SOC (interferon sparing).

1. A method of treatment for treating or diminishing hepatitis C (HepC) infection in a target subject, comprising administering to said target subject an effective amount of an immunomodulator compound of Formula A 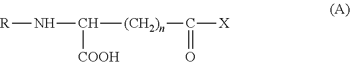
wherein, n is 1 or 2, R is hydrogen, acyl, alkyl or a peptide fragment, and X is an aromatic or heterocyclic amino acid or a derivative thereof, wherein;
(i) said immunomodulator compound is administered to said subject at a dosage of greater than 0.001 mg/kg;
(ii) said immunomodulator compound is administered in a combination treatment regimen further comprising administration to said subject of ribavirin, wherein said immunomodulator compound and said ribavirin are administered to the subject separately or together in the treatment regimen;
(iii) said immunomodulator compound is administered in a combination treatment regimen with a specifically targeted antiviral therapy for hepatitis C (STAT-C) agent, wherein the immunomodulator compound and the STAT-C agent are administered to the subject separately or together in the treatment regimen; or
(iv) a combination of at least two of (i), (ii), and (iii).

2. The method of claim 1, wherein X is L-tryptophan or D-tryptophan.

3. The method of claim 1 wherein said immunomodulator compound is SCV-07.

4. The method of claim 1 wherein, said immunomodulator compound is administered at a dosage within a range of from greater than 0.001 mg up to about 700 mg.

5. The method of claim 4 wherein said range is about 0.07-700 mg.

6. The method of claim 4 wherein said range is about 0.7-70 mg.

7. The method of claim 4 wherein said range is about 7-70 mg.

8. The method of claim 1 wherein said immunomodulator compound is administered at a dosage within a range of from greater than 0.001 mg/kg up to about 10 mg/kg.

9. The method of claim 8 wherein said range is from greater than about 0.01 mg/kg to about 10 mg/kg.

10. The method of claim 8 wherein said range is about 0.1-1 mg/kg.

11. The method of claim 1 wherein, in said combination treatment regimen, said ribavirin is administered at a dosage within a range of about 100-2000 mg/day.

12. The method of claim 11 wherein said range is about 800-1200 mg/day.

13. The method of claim 11 wherein said range is about 1000-1200 mg/day.

14. The method of claim 1 wherein said immunomodulator compound administered to said target subject in a treatment regimen without administering an interferon to said target subject.

15. The method of claim 14 wherein said at least one interferon is an alpha interferon, a beta interferon, a gamma interferon or a combination thereof.

16. The method of claim 14 wherein said interferon is an alpha interferon.

17. The method of claim 14 wherein said interferon is a beta interferon.

18. The method of claim 14 wherein said interferon is a gamma interferon.

19. The method of claim 1 wherein said STAT-C agent is administered at a dosage of about 1000-3000 mg/day.

20. The method of claim 1 wherein, in said treatment regimen, ribavirin is administered at a dosage within a range of about 100-2000 mg/day, either separately or together with at lease one of the Formula A compounds or the STAT-C agent.

21. The method of claim 1 wherein said STAT-C agent comprises SCH503034, VX-950, VX500, R7227, ITMN-191, ACH-1095, TMC435350, BILN-2061, ACH-806, GS9190, GSK625433, R7128, R6126, VCH-759, MK-0608, IDX-184, A-837093, AG-021541, NM283, HCV-796 or a combination thereof.

22. The method of claim 1 wherein at least one interferon is administered with said immunomodulator compound, wherein the immunomodulator compound and the interferon are administered to the subject separately or together in a treatment regimen.

23. The method of claim 22 wherein said interferon is interferon alpha-2a.

24. The method of claim 23 wherein said interferon is pegylated interferon alpha-2a.

25. The method of claim 22 wherein said interferon is interferon alpha-2b.

26. The method of claim 25 wherein said interferon is pegylated interferon alpha-2b.


SEQUENCE LISTING (8 sequences)

SEQ ID NO: 1 - protein, 2 aa, Artificial Sequence
  synthetic oligopeptide with immunomodulating activity toward Hepatitis C virus
XW

SEQ ID NO: 2 - protein, 2 aa, Artificial Sequence
  synthetic oligopeptide with immunomodulating activity toward Hepatitis C virus
EW

SEQ ID NO: 3 - protein, 3 aa, Artificial Sequence
  synthetic oligopeptide with immunomodulating activity toward Hepatitis C virus
EXW

SEQ ID NO: 4 - protein, 3 aa, Artificial Sequence
  synthetic oligopeptide with immunomodulating activity toward Hepatitis C virus
MEW

SEQ ID NO: 5 - protein, 3 aa, Artificial Sequence
  synthetic oligopeptide with immunomodulating activity toward Hepatitis C virus
XEW

SEQ ID NO: 6 - protein, 2 aa, Artificial Sequence
  synthetic oligopeptide with immunomodulating activity toward Hepatitis C virus
EX

SEQ ID NO: 7 - protein, 2 aa, Artificial Sequence
  synthetic oligopeptide with immunomodulating activity toward Hepatitis C virus
DW

SEQ ID NO: 8 - protein, 2 aa, Artificial Sequence
  synthetic oligopeptide with immunomodulating activity toward Hepatitis C virus
XW